(19) United States
(12) Patent Application Publication
(10) Pub. No.: US 2005/0070993 A1
(43) Pub. Date: Mar. 31, 2005

(54) Methods of retroperfusion and related devices

(76) Inventors: Peter Boekstegers, Diessen (DE); Peter Wilk, New York, NY (US); Laurence Roth, Windham, NH (US); Jianlu Ma, Merrimack, NH (US); Earl Bardsley, Newton, MA (US)

Correspondence Address: Susanne T. Jones;FINNEGAN, HENDERSON, FARABOW,, GARRETT & DUNNER, L.L.P, 1300 I Street, N.W., Washington, DC, 20002-3315, US

(21) Appl. No.: 10/671,581

(22) Filed: Sep. 29, 2003

(51) Int. Cl. A61F 2/06

(52) U.S. Cl. 623/1.25; 128/898; 606/158; 623/1.24; 623/1.31

(57) ABSTRACT

Methods, and related devices, for treating a heart by allowing retrograde blood flow in a coronary vein may include flowing blood through a passageway between a blood-containing anatomical structure and a coronary vein so as to cause retrograde blood flow in the coronary vein. The methods, and related devices, may further include measuring an internal pressure of the coronary vein to determine a measured internal pressure and at least partially obstructing the coronary vein at a location upstream of the passageway relative to a direction of the retrograde blood flow based on the measured internal pressure.

### BACKGROUND OF THE INVENTION

1. Field of the Invention

The present invention relates to methods and related devices for supplying fluid flow to the venous system in a retrograde manner. For example, the methods and related devices allow retrograde oxygenated blood flow in a coronary vein, for example, by forming a passageway between a blood-containing anatomical structure, which may contain oxygenated blood, such as a left ventricle or coronary artery, for example, and the coronary vein and controlling internal pressure of the coronary vein during the retroperfusion by at least partially obstructing the coronary vein at a location upstream of the passageway.

2. Background of the Related Art

A prevalent form of heart failure involves the build-up of plaque on walls of various vascular structure, such as, for example, the coronary artery. The plaque that builds up on the walls can form either a partial or total occlusion in the artery. Such an occlusion may either limit or completely block blood flow through the artery, which typically enters the artery through the aortic valve from the left ventricle. Because the coronary artery supplies blood to the various blood vessels within the muscle forming the heart wall, limiting or blocking the blood flow through the coronary artery can result in damage to the heart muscle, such as, for example, ischemia. Ischemic tissue can lead to reduced cardiac function by diminishing the pumping capacity of the heart. In some instances, the diminished capacity of the heart can lead to heart attack.

Various techniques have been developed to treat this type of heart condition. For example, a surgical technique, referred to as coronary artery bypass grafting (CABG), involves removing a vein or portion thereof from the patient, usually from the femoral vein, and grafting the vein so as to connect portions of the coronary artery upstream and downstream of the occlusion. Thus, the blood flow is diverted around the occlusion and through the vein graft so that the oxygenated blood can be delivered to the vessels in the heart wall. CABG generally is performed as an open surgery resulting in relatively long recovery times. Patients also often experience a large amount of discomfort resulting from harvesting of the veins to be used in CABG. Furthermore, the veins that are grafted to the coronary artery have a limited useful life.

Coronary angioplasty represents another form of treatment of arteries having occlusions that can be performed as an alternative to bypass surgery. In this technique, a balloon catheter is inserted percutaneously into the coronary artery. Once the catheter has been inserted such that the balloon is adjacent the occlusion being treated, the balloon is inflated to dilate the artery in the location of the occlusion. Often this technique involves inflating and deflating the balloon repeatedly to establish the desired dilation of the artery. This technique may include placing a stent in a collinear manner in the artery at the location of the occlusion to maintain the proper dilation of the artery. Delivery of the stent can be accomplished by removing the dilation balloon catheter and then inserting a balloon catheter carrying the stent into the artery. A multiple balloon stent delivery catheter may dilate the artery and place the stent in a single insertion of the catheter into the patient.

Yet another technique includes delivering oxygenated blood to the heart wall in a retrograde manner by creating a passageway between a coronary artery and a coronary vein at a location upstream of the occlusion in the coronary artery. In this technique the coronary vein is typically ligated or otherwise blocked to prevent antegrade blood flow from the passageway into the coronary sinus. The arterial blood is thus introduced from the coronary artery, through the passageway, and into the coronary vein where it may flow in a retrograde manner to perfuse the heart wall. This retroperfusion technique (i.e. using retrograde flow in the venous system from the arterial system to perfuse the heart), however, may be associated with relatively high incidences of hemorrhagic infarct or tissue engorgement leading to cell death. Such problems may be caused at least in part by preventing antegrade blood flow into the coronary sinus. In other words, under certain conditions, performing retroperfusion without permitting the an outflow from the heart through the venous system may cause overperfusion of the heart.

Moreover, conventional techniques to retroperfuse the heart by delivering blood from a coronary artery to the coronary vein in a retrograde manner have not taken into account the internal pressure experienced by the coronary vein, which as explained above may have effects on both the vein itself as well as well as the ability to perfuse the heart. For example, completely blocking or ligating the coronary vein proximate the location of the passageway, in addition to causing overperfusion of the heart, may potentially damage the vein due to increased pressures in the vein above those found under normal conditions (i.e., in the absence of arterial retrograde blood flow).

On the other hand, however, to leave the vein unblocked may result in inefficient perfusion of the heart due to competition between the retrograde blood flow through the vein with the antegrade blood flow through the vein. Such competing flows may result in an the heart being underperfused.

It may thus be desirable to provide a technique for retroperfusing the heart that permits at least some antegrade blood flow through the venous system.

It also may be desirable to provide a retroperfusion technique that controls pressure in the vessel, for example, limiting pressure generated in the vessel, through which retrograde blood flow occurs. For example, it may be desirable to limit the pressure such that it is maintained substantially at or below a predetermined value or predetermined range of values, which may be selected based on a measured internal pressure of the coronary vein.

### SUMMARY OF THE INVENTION

It should be understood that the invention could be practiced without performing one or more of the aspects, objects, or advantages described above. Other aspects will become apparent from the following detailed description.

An aspect of the invention includes a method of treating a heart that includes flowing blood through a passageway between a blood-containing anatomical structure, for example an oxygenated blood-containing anatomical structure, and a coronary vein so as to cause retrograde blood flow in the coronary vein. The method further includes measuring an internal pressure of the coronary vein to determine a measured internal pressure. The method may also include at least partially obstructing the coronary vein upstream, with respect to the retrograde blood flow, of the passageway based on the measured internal pressure.

At least partially obstructing may include (1) allowing at least some antegrade blood flow past the location throughout a cardiac cycle, (2) allowing at least some antegrade blood flow past the location during a portion a cardiac cycle and preventing antegrade blood flow past the location during another portion a cardiac cycle, or (3) completely obstructing the vessel so as to prevent antegrade blood flow past the location throughout a cardiac cycle.

Another aspect of the invention includes a method of treating a heart comprising flowing blood through a passageway between a heart chamber and a coronary vein so as to cause retrograde blood flow in the coronary vein and at least partially obstructing the coronary vein at a location upstream of the passageway relative to a direction of the retrograde blood flow.

Yet another aspect of the invention includes a system for use in retroperfusing a heart by causing retrograde blood flow in a coronary vein. The system may include a pressure measuring device configured to measure the internal pressure of a coronary vein. The system also may include an implant configured to be placed relative to the coronary vein at a location upstream, relative to a direction of the retrograde blood flow, of a source producing the retrograde blood flow. The implant may be configured to at least partially obstruct the coronary vein.

Yet another exemplary aspect of the invention includes a device for use in retroperfusing a heart by causing retrograde blood flow in a coronary vein. The device includes an implant configured to be placed within the coronary vein at a location upstream, relative to a direction of the retrograde blood flow, of a passageway between the coronary vein and a heart chamber. The implant may be configured to at least partially obstruct the coronary vein.

Aside from the structural and procedural arrangements set forth above, the invention could include a number of other arrangements, such as those explained hereinafter. It is to be understood that both the foregoing general description and the following detailed description are exemplary and explanatory only and are not restrictive of the invention.

### BRIEF DESCRIPTION OF THE DRAWINGS

The accompanying drawings are included to provide a further understanding of the invention and are incorporated in and constitute a part of this specification. The drawings illustrate exemplary embodiments of the invention, and together with the description, serve to explain certain principles. In the drawings,

FIG. 1 is a partial cross-sectional view of a heart with a passageway formed between a left ventricle and a coronary vein and a blocking device positioned in the vein according to an exemplary embodiment;

FIG. 2 is a partial cross-sectional view of a heart with a passageway formed between a coronary artery and a coronary vein and a blocking device positioned in the vein according to an exemplary embodiment;

FIG. 3 is a partial, cross-sectional view of a left ventricle and coronary vein with an exemplary embodiment of a blocking device for completely obstructing antegrade flow in the coronary vein;

FIGS. 4A and 4B are partial, cross-sectional views of a left ventricle and coronary vein with an exemplary embodiment of a blocking device for at least partially obstructing antegrade flow in the coronary vein;

FIG. 5A is a partial, cross-sectional view of a left ventricle and coronary vein with an exemplary embodiment of a blocking device for at least partially obstructing antegrade blood flow in the coronary vein;

FIG. 5B is a partial, cross-sectional view of a left ventricle and coronary vein with an exemplary embodiment of a blocking device shown;

FIGS. 5C and 5D are partial, cross-sectional views of a left ventricle and coronary vein with an exemplary embodiment of a blocking device for obstructing at least some antegrade flow in the coronary vein and shown during differing portions of the cardiac cycle;

FIG. 6 is a partial, cross-sectional view of a left ventricle and coronary vein with another exemplary embodiment of a blocking device for at least partially obstructing antegrade blood flow in the coronary vein;

FIG. 7 is a partial, cross-sectional view of a left ventricle and coronary vein with an exemplary embodiment of a blocking device for at least partially obstructing antegrade blood flow in the coronary vein;

FIG. 8 is a partial, cross-sectional view of a left ventricle and coronary vein with yet another exemplary embodiment of a blocking device for at least partially obstructing antegrade blood flow in the coronary vein;

FIG. 9 is a partial, cross-sectional view of a left ventricle and coronary vein with another exemplary embodiment of a blocking device for at least partially obstructing antegrade blood flow in the coronary vein;

FIG. 10A is a perspective view of yet another exemplary embodiment of a blocking device in an expanded, deployed configuration for at least partially obstructing antegrade blood flow in the coronary vein;

FIG. 10B is a partial, perspective view of the restricted region of the blocking device of FIG. 10A;

FIG. 10C is a partial, perspective view of a portion other than the restricted region of the blocking device of FIG. 10A;

FIG. 10D is a partial, perspective view of the blocking device of FIG. 10A including a covering;

FIG. 11 is a partial, cross-sectional view of a left ventricle and coronary vein with another exemplary embodiment of a blocking device for at least partially obstructing antegrade blood flow in the coronary vein;

FIGS. 12A and 12B are partial, cross-sectional views of a left ventricle and coronary vein with another exemplary embodiment of a blocking device for at least partially obstructing antegrade flow in the coronary vein shown during differing portions of the cardiac cycle;

FIG. 13 is a partial, cross-sectional view of a left ventricle and coronary vein with another exemplary embodiment of a blocking device for at least partially obstructing antegrade blood flow in the coronary vein;

FIGS. 14A and 14B are partial, cross-sectional views of a left ventricle and coronary vein with yet another exemplary embodiment of a blocking device for at least partially obstructing antegrade flow in the coronary vein shown during differing portions of the cardiac cycle; and

FIG. 15 is a partial, cross-sectional view of a left ventricle and coronary vein with another exemplary embodiment of a blocking device for at least partially obstructing antegrade blood flow in the coronary vein.

### DESCRIPTION OF EXEMPLARY EMBODIMENTS

The present invention pertains to methods, and related devices for performing the methods, of retroperfusing a heart by flowing oxygenated blood in the venous system in a retrograde manner. By way of example, the disclosed methods and devices may be used to bypass a total or partial occlusion of a coronary vessel by forming a passageway between a heart chamber (e.g., left ventricle) and a vein (e.g., coronary vein) or between an artery (e.g., coronary artery) and a vein (e.g., coronary vein) and allowing retrograde blood flow in the vein to at least a portion of a heart wall. The techniques using the methods and devices may be performed either surgically or via a less invasive technique, such as percutaneous approaches, for example.

The method may generally include flowing blood through a passageway between a coronary vein and an oxygenated blood-containing anatomical structure so as to allow retrograde blood flow in the coronary vein and controlling the internal pressure of the coronary vein during the retroperfusion by at least partially obstructing the coronary vein at a location upstream of the passageway. When used herein, the terms “upstream” and “downstream” are relative to the direction of the retrograde blood flow in the vein, unless otherwise indicated. The method may include measuring an internal pressure of the coronary venous system via a pressure measuring device and at least partially obstructing the coronary vein based on the measured internal pressure. The at least partially obstructing the coronary vein based on the measured internal pressure may include, among other things, selecting an amount of obstruction and/or location of the obstruction.

By way of example, the internal pressure that is measured in the coronary venous system is the wedge pressure, and more particularly, the mean wedge pressure, for example.

Although the methods and devices according to exemplary aspects of the present invention will be described with reference to retroperfusing a heart by forming a passageway between an oxygenated blood-containing anatomical structure (e.g., a heart chamber, such as a left ventricle, or a coronary vessel, such as a coronary artery) and the coronary vein to allow retrograde blood flow in the coronary vein, the methods and devices described may be practiced in various other settings, aside from the heart, to provide blood flow to a target area in a retrograde manner. Further, oxygenated blood-containing anatomical structures may be any such structure, including other heart chambers, other coronary vessels, and any other oxygenated blood-containing anatomical structures. In addition, the source of oxygenated blood for providing the retrograde blood flow to the coronary vein may be any other type of source, whether natural or artificial.

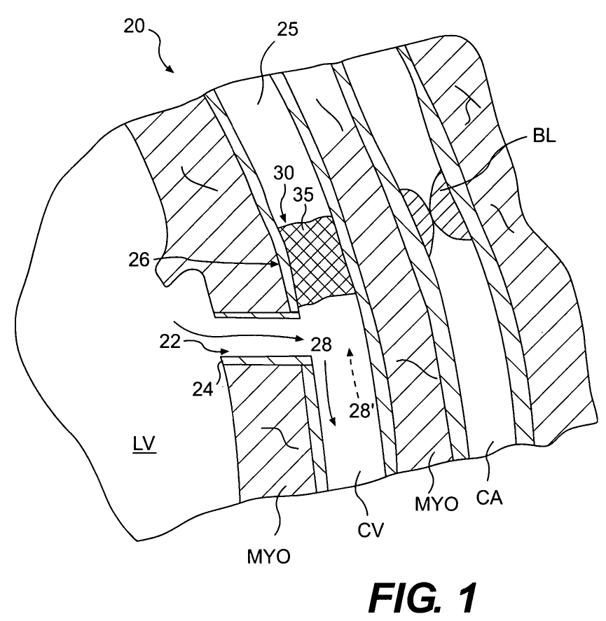

FIG. 1 shows a partial cross-sectional view of a heart 20 with a passageway 22 formed between a left ventricle LV and a coronary vein CV. The passageway 22 is formed through the myocardium MYO. The passageway 22 is formed at a desired location, such as at a location on either side of a blockage BL in a coronary artery CA. In an exemplary aspect, a conduit 24 may be placed in the passageway 22 as shown. The passageway 22 establishes oxygenated blood flow between the left ventricle LV and the coronary vein CV and allows retrograde blood flow 28 (i.e., blood flow opposite to a usual, antegrade direction 28′ shown by a dashed line) in the coronary vein CV. The retrograde blood flow 28 may thereby perfuse the myocardium MYO, which may be deprived of oxygenated blood due to the blockage BL in the coronary artery CA. Although the blockage BL is shown in the figures as a total blockage, it should be understood that the methods and devices described herein may also be used to treat partial blockages.

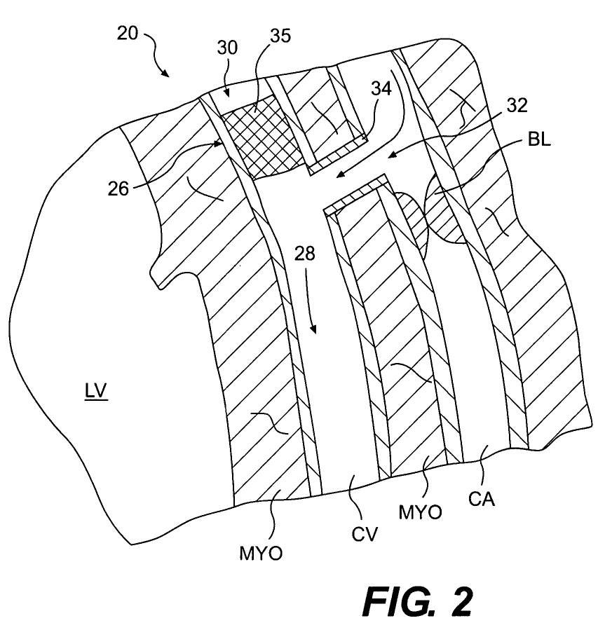

FIG. 2 shows another exemplary way of retroperfusing a heart. In FIG. 2, a passageway 32 is formed between the coronary artery CA and the coronary vein CV at a point proximal (e.g. upstream with respect to the flow through the coronary artery CA) the blockage BL. The passageway 32 may be formed through the myocardium MYO, as shown in FIG. 2. Alternatively, the passageway 32 may be formed directly between the coronary artery CA and the coronary CV. A conduit 34 may be placed in the passageway 32 as shown. The passageway 32 establishes oxygenated blood flow between the coronary artery CA and the coronary vein CV and allows retrograde blood flow 28 in the coronary vein CV. The retrograde blood flow 28 may thereby perfuse the myocardium MYO, which may be deprived of oxygenated blood due to the blockage BL in the coronary artery CA.

To facilitate the retrograde blood flow 28, the coronary vein CV may be at least partially obstructed at a location 26, as illustrated in FIGS. 1 and 2. The location 26 is upstream of the passageway relative to the direction of the retrograde blood flow 28. As explained in greater detail below, the obstruction at the location 26 may be a complete obstruction that prevents antegrade blood flow (i.e., blood flow in a usual direction and opposite the retrograde flow direction) past the location 26 throughout a cardiac cycle. On the other hand, the obstruction may allow at least some antegrade blood flow past the location 26 during a least a portion of a cardiac cycle. For example, the obstruction may allow at least some antegrade blood flow past the location throughout the cardiac cycle or may allow at least some antegrade blood flow past the location during only a portion of a cardiac cycle while preventing antegrade blood flow past the location during another portion of the cardiac cycle.

As illustrated in FIGS. 1 and 2, retroperfusion of the heart may be performed by forming a passageway between a left ventricle and a coronary vein (ventricle-to-vein) or between a coronary artery and a coronary vein (artery-to-vein) and at least partially obstructing the coronary vein at a location upstream of the passageway, for example. Although the following description explains other aspects of the inventions with reference to a ventricle-to-vein retroperfusion, they are equally applicable to an artery-to-vein retroperfusion as well. Also, it should be noted that the methods of present invention may be practiced to bypass and treat blockages that totally obstruct blood flow in the coronary artery or that partially obstruct blood flow in the coronary artery CA.

In accordance with an exemplary aspect, the internal pressure of the coronary vein CV during retroperfusion may be controlled. The internal pressure of the coronary vein CV during retroperfusion should be high enough to cause adequate retrograde blood flow to the ischemic region of the heart being treated. For example, the pressure should be such that the retrograde blood flow is sufficient to overcome the antegrade blood flow through the vein so that the heart is not underperfused. At the same time, the internal pressure of the coronary vein CV during retroperfusion should not be too high so as to cause overperfusion of the heart and/or potential damage to the coronary vein CV. An optimal value, or a range of values, for the internal pressure of the coronary vein CV during retroperfusion may vary depending on the particular patient, the particular coronary vein in which the retroperfusion is to be performed, and the particular site of the retroperfusion in the coronary vein.

Accordingly, the internal pressure, such as the mean wedge pressure, for example, of the coronary venous system may be measured, and an amount of obstruction of antegrade flow in the vein may be tailored for a particular patient and selected based on the measured pressure so as to achieve and maintain as closely as possible a predetermined pressure or range of pressures. The mean wedge pressure of the coronary venous system may be measured before establishing the retrograde blood flow therethrough, for example, before forming the passageway 22. Alternatively, the mean wedge pressure may be measured after forming the passageway 22. An exemplary method of measuring mean wedge pressure of the coronary vein CV and determining a target pressure range for the coronary vein while establishing retrograde flow in the vein is disclosed in U.S. Pat. No. 6,458,323 to Boekstegers. The entire disclosure of U.S. Pat. No. 6,458,323 is incorporated herein by reference.

By way of example, pressure measurement may be performed by inserting a balloon or other type of occluder into the vein and measuring the pressure via a pressure transducer or pressure-measuring guidewire distal to the occluder. Such pressure measurement techniques would be obvious to those skilled in the art. As mentioned above, it also is contemplated that the pressure could be measured after formation of the passageway, in which case the mean wedge pressure could be measured using similar techniques in order to determine a desired amount and/or location of the obstruction.

In addition to the amount of obstruction, the location of the obstruction may be selected to control the internal pressure of the coronary vein CV during retroperfusion. For example, given the same amount of obstruction, selecting the location of the obstruction closer to the passageway 22 may reduce the internal pressure of the coronary vein CV during retroperfusion. Accordingly, the internal pressure of the coronary vessel during retroperfusion may be controlled by selecting the amount of the obstruction, selecting the location of the obstruction, or selecting the combination of the amount and the location of the obstruction.

Aside from the amount of obstruction and/or location of the obstruction, other mechanisms, such as pressure absorption mechanisms, for example, also may be used to control the internal pressure of the coronary vein. Examples of such mechanisms for controlling the internal pressure of the coronary vein are described in more detail below.

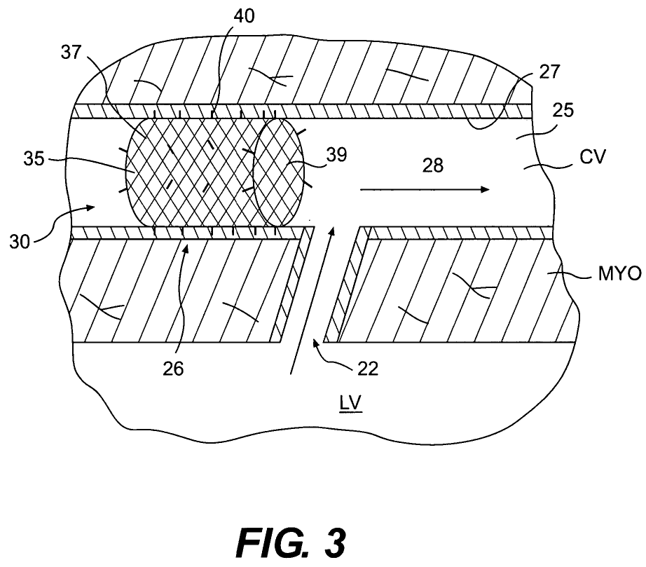

According to an exemplary aspect, the coronary vein CV may be completely obstructed at the location 26 so as to prevent antegrade blood flow past the location 26 throughout a cardiac cycle. FIG. 3 illustrates an exemplary embodiment of a blocking device for completely obstructing the coronary vein. As shown, the blocking device 30 is in the form of a completely-obstructing implant 35, which may have a stent-like structure, for example, to be placed within a lumen 25 of the coronary vein CV at the location 26. The completely-obstructing implant 35 includes an external surface 37 configured to engage an inner surface 27 of the coronary vein CV and an end face 39, which may disposed so as to face the passageway 22, as shown in FIG. 3, for example. The end face 39 may be substantially rigid so that it does not deform substantially throughout a cardiac cycle. Accordingly, if the completely-obstructing implant 35 is used, the internal pressure of the coronary vein CV during retroperfusion may be controlled by selecting the location 26 of the implant 35 relative to the passageway 22.

The completely-obstructing implant 35 may be self-expandable. It may be delivered to the location 26 via a catheter or sheath (not shown) in a collapsed state and may be pushed out of the catheter or sheath into the lumen 25 of the coronary vein CV. Upon being pushed out of the catheter or sheath, the completely-obstructing implant 35 may expand to engage its external surface 37 with the inner surface 27 of the coronary vein CV. Alternatively, the implant may be balloon expandable and delivered via a balloon catheter, for example.

It is envisioned that the implant 35 may be a substantially hollow structure or it may be a solid structure. In either case, the implant 35 has an end face 39 which blocks flow through the implant 35. Skilled artisans would understand that the impermeable end face 39 could be positioned at either or both ends of the implant 35.

To help secure the implant 35 within the lumen 25 at the location 26, an attachment mechanism may be provided on the implant 35. As shown in FIG. 3, the attachment mechanism may be in the form of a plurality of prongs 40 extending from the external surface 37. Alternatively or in addition to the prongs 40, the implant 35 may have a collar, a flange, tabs, barbs, or other similar attachment mechanisms surrounding its external surface to help maintain the implant 35 in position within the vein.

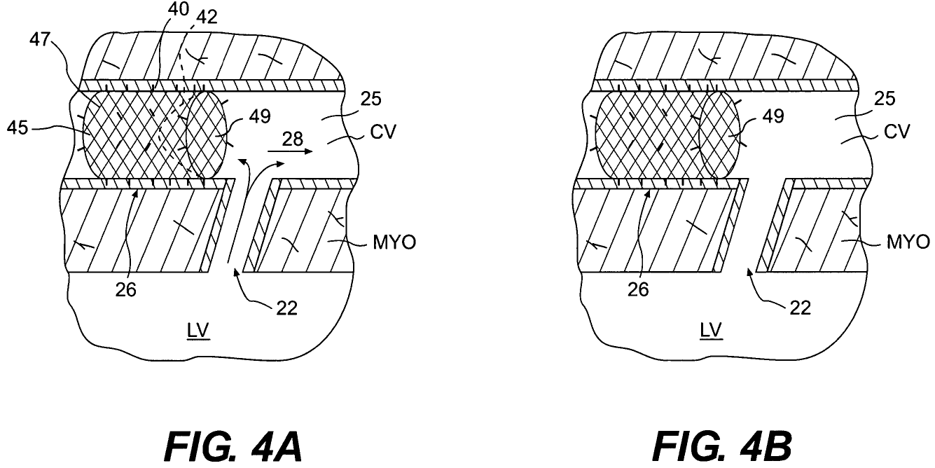

FIGS. 4A and 4B illustrate another exemplary embodiment of a completely-obstructing implant to be placed within the lumen of the coronary vein. The completely-obstructing implant 45 illustrated in FIGS. 4A and 4B is substantially the same as the embodiment illustrated in FIG. 3 except for the end face 49. Rather than being substantially rigid, the end face 49 is flexible so as to permit deformation of the end face 49. For example, during diastole, the end face 49 may deform, as designated by the dotted lines and reference numeral 42 in FIG. 4A, toward the opposite end portion of the implant 45, thereby accommodating some of the blood flowing into the coronary vein CV from the passageway 22. This deformation may permit some antegrade blood flow in the coronary vein CV upstream of the passageway 22 and toward the location 26 and also may absorb some pressure that otherwise may build up in the coronary vein CV due to completely obstructing the vein. During systole, on the other hand, the end face 49 regains its original shape.

The deformation of the end face 49 may provide a mechanism by which to control the internal pressure of the coronary vein CV during retroperfusion. Accordingly, if the completely-obstructing implant 45 with the flexible end face 49 is used, the internal pressure of the coronary vein CV during retroperfusion may be controlled by selecting the location 26 relative to the passageway 22 and/or selecting an appropriate flexibility, and thus degree of deformation, of the end face 49. Like the exemplary embodiment of FIG. 3, the completely-obstructing implant 45 may be self-expandable and may include an attachment mechanism, such as, for example, a plurality of prongs 40 extending from the external surface 47, and/or other suitable attachment mechanisms. Rather than being self-expandable, it is contemplated that the implants of FIGS. 3, 4A, and 4B may be expandable via balloons and other dilation devices.

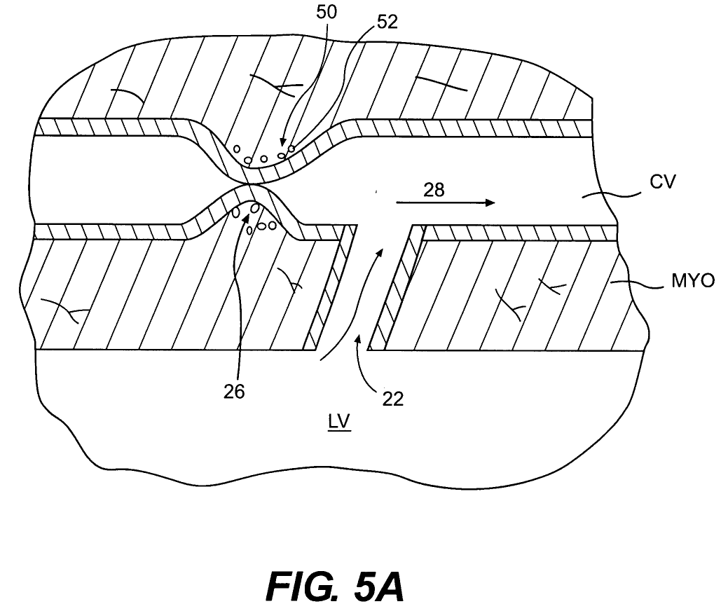

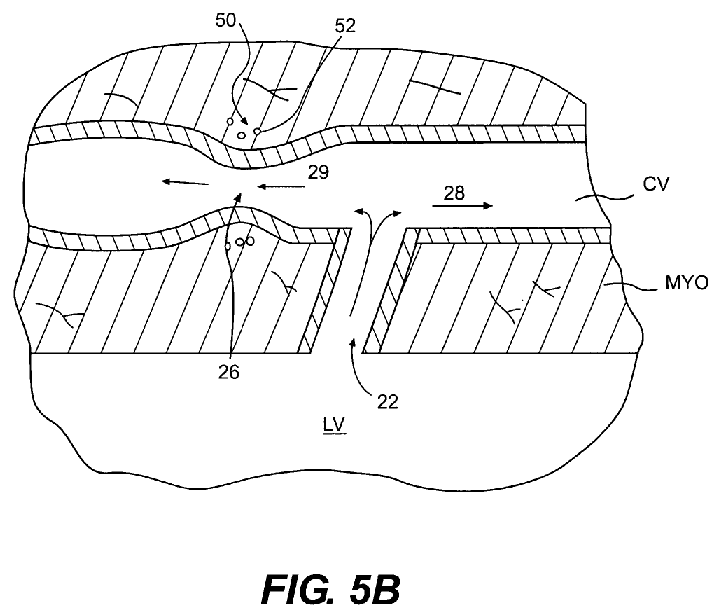

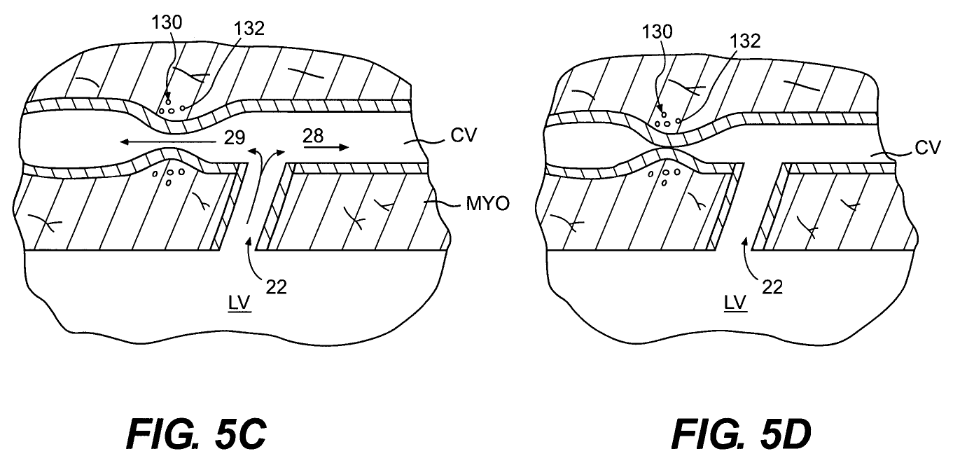

Yet another exemplary embodiment of a device for obstructing the coronary vein through which retrograde blood flow occurs is shown in FIGS. 5A and 5B. In FIG. 5A, the blocking device 50 is in the form of a plurality of magnetic elements 52 configured to completely collapse the coronary vein CV at the location 26. In FIG. 5B, on the other hand, the blocking device 50 is in the form of a plurality of magnetic elements 52 configured to partially collapse the coronary vein CV at the location 26. The magnetic elements 52 may be placed in tissue surrounding the coronary vein CV, as shown in FIG. 5A, and their magnetic attraction may be such as to either partially or completely collapse the coronary vein CV at the location 26.

Alternatively, or in addition to, the magnetic elements 52 may be placed in or attached to the walls of the coronary vein CV (not shown). The number and/or the magnetic strength of the magnetic elements 52 may be appropriately selected to ensure that the coronary vein CV is either partially or completely collapsed at the location 26, as desired. Accordingly, if the number and/or the magnetic strength of the magnetic elements 52 are selected to completely collapse the coronary vein CV (FIG. 5A), the internal pressure of the coronary vein CV during retroperfusion may be controlled by selecting the location 26 of the magnetic elements 52 relative to the passageway 22. On the other hand, if the magnetic elements 52 are such that the coronary vein CV only partially collapses, allowing at least some antegrade blood flow past the location 26, the field strength of the magnetic elements 52 and/or the number of magnetic elements 52 may be chosen so as to control the amount by which the coronary vein CV is collapsed in order to in turn control the internal pressure of the coronary vein CV during retroperfusion. As described above, these features to control the internal pressure of the coronary vein CV during retroperfusion may be selected based on the measured internal pressure, for example mean wedge pressure, of the coronary vein CV before or after forming the passageway 22. Using the plurality of magnetic elements 52 as shown in FIGS. 5A and 5B may be beneficial in that the elements 52 are not in contact with blood flowing in the vein. This may reduce the risk of thrombus formation.

Exemplary magnetic elements and methods of placing magnetic elements into body structures are disclosed in U.S. Patent Application Publication No. 2000/0091295 A1, entitled “Medical Treatment Method and Device Utilizing Magnetic Particles.” The entire disclosure of U.S. Patent Application Publication No. 2000/0091295 A1 is incorporated herein by reference.

As mentioned above, rather than being completely obstructed at the location 26, the coronary vein CV may be partially obstructed at the location 26 so as to allow at least some antegrade blood flow past the location 26 during at least a portion of a cardiac cycle. For example, a partial obstruction may allow at least some antegrade blood flow past the location throughout a cardiac cycle. Alternatively, a partial obstruction may allow at least some antegrade blood flow past the location during a portion of a cardiac cycle and prevent antegrade blood flow past the location during another portion of the cardiac cycle.

FIGS. 5C and 5D illustrate an exemplary embodiment of a blocking device formed of a plurality of magnetic elements which allows at least some antegrade blood flow past the location during a portion of the cardiac cycle and prevents antegrade blood flow during another portion of the cardiac cycle. This embodiment is similar to the embodiment illustrated in FIGS. 5A and 5B, except that the number, and/or the magnetic strength, and/or the location of the magnetic elements 132 are appropriately selected such that the coronary vein CV at the location 26 is partially collapsed during a first portion of the cardiac cycle, as shown in FIG. 5C, and completely collapsed during a second portion of the cardiac cycle, as shown in FIG. 5D. By way of an example, it may be desirable to control the magnetic elements 132 such that the coronary vein CV partially collapses during systole, permitting at least some antegrade blood flow past location 26, and completely collapses during diastole, preventing antegrade blood flow past location 26.

To control the collapsing of the vein in conjunction with the various portions of the cardiac cycle, the magnetic elements 132 may be influenced by electrical signals associated with the heart during the various portions of the cardiac cycle, for example. Alternatively, an externally controlled magnetic source (not shown) may be coupled to the magnets 132 and may either automatically or manually be activated to cause the magnetic strength of the magnets 132 to change depending on the desired degree of collapsing of the vein during a particular portion of the cardiac cycle. Other means for controlling the degree of collapse of the vein and timing the collapsing of the vein with portions of the cardiac cycle may also be used and are considered within the scope of this invention.

Accordingly, in addition to the location 26, the number and/or the magnetic strength of the magnetic elements 132, in addition to altering the magnetic strength depending on the phase of the cardiac cycle, may be selected to control the internal pressure of the coronary vein CV during retroperfusion. As described above, these various features to control the internal pressure of the coronary vein CV during retroperfusion may be selected based on the measured internal pressure of the coronary vein CV before or after forming the passageway 22.

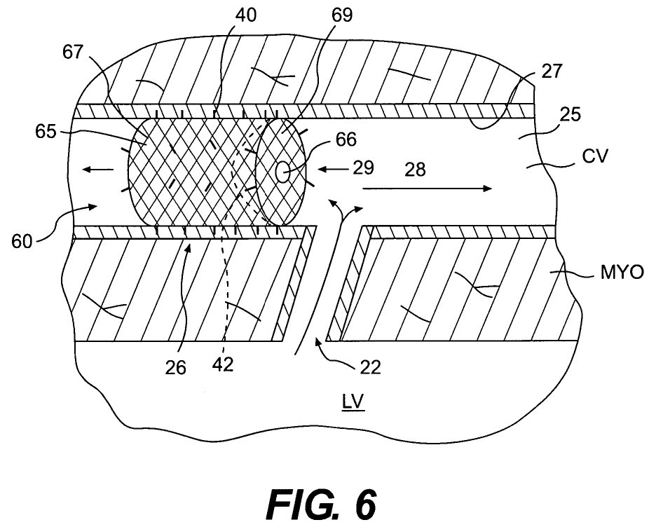

FIG. 6 illustrates an exemplary embodiment of a blocking device for partially obstructing the coronary vein at a location so as to allow antegrade blood flow past the location. As shown, the blocking device 60 is in the form of a partially-obstructing implant 65 configured to be placed within the lumen 25 of the coronary vein CV. The partially-obstructing implant 65 includes an external surface 67 configured to engage the inner surface 27 of the coronary vein CV and an end face 69. The end face 69 may face the passageway 22 and may be substantially rigid so that it does not deform throughout the cardiac cycle. Alternatively, as described with respect to FIGS. 4A and 4B, the end face 69 may be flexible, allowing deformation. For example, the end face 69 may deform toward the opposite end portion throughout the cardiac cycle or during only a portion of the cardiac cycle. In the latter case, for example, the end face 69 may be configured so as to deform during diastole and/or during systole. According to an exemplary embodiment, when the end face 69 is configured so as to deform during systole, it may be possible to reduce systolic peak pressure in the vein, which in turn may assist in efficient perfusion of the heart wall.

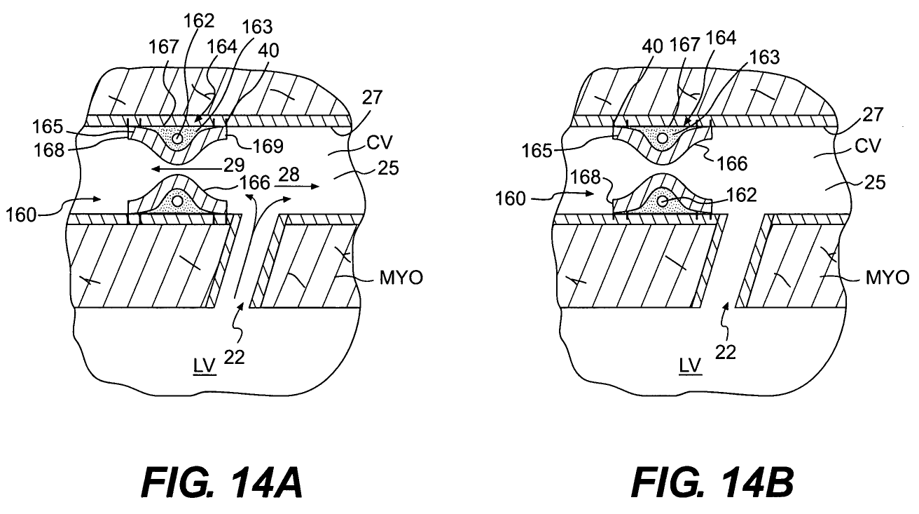

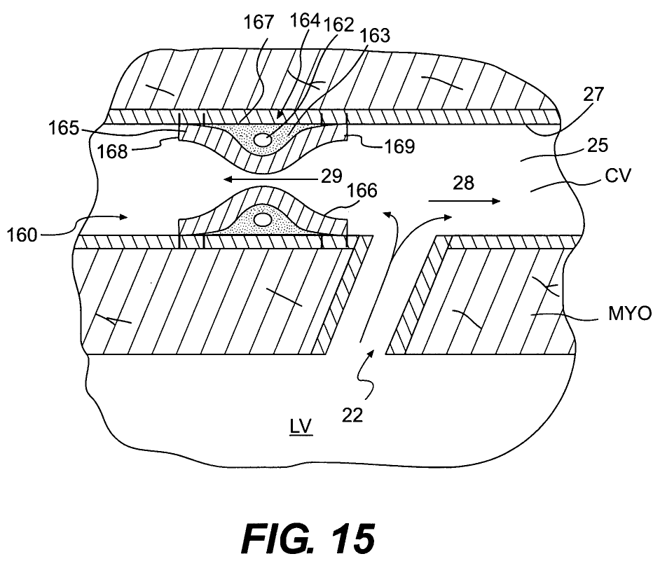

The partially-obstructing implant 65 further defines a passage 66 configured to allow at least some antegrade blood flow 29 therethrough. This passage 66 may be configured to allow some antegrade blood flow therethrough throughout a cardiac cycle. Alternatively, the passage 66 may be configured to close during at least part of the cardiac cycle thereby allowing antegrade blood flow through the passage 66 during only part of the cardiac cycle. For example, the passage 66 may be configured to close (for example, by collapse of the implant 65) during systole and open to permit antegrade blood flow therethrough during diastole. Such deformation may occur due to the structure of the implant 60 itself, which may take into account the various forces experienced by the vein during the cardiac cycle and be configured so as to collapse and open, respectively, in conjunction with those forces. Alternatively, an external source may be used to control the collapsing and opening of the implant. Exemplary embodiments of such devices to control the collapse and opening of an implant having similar structures to the implant 60 are described below with reference to FIGS. 14A, 14B, and 15. However, other means for controlling the collapse and opening of the implant 60 are also considered within the scope of the invention.

In the illustrated embodiment of FIG. 6, the passage 66 has a substantially uniform cross-section. Accordingly, in addition to the location 26, the cross-section and the length of the passage 66 may be selected so as to control the internal pressure of the coronary vein CV during retroperfusion. If a partially-obstructing implant 65 having a flexible end portion 69 is used, the internal pressure of the coronary vein CV during retroperfusion may further be controlled by selecting appropriate flexibility of the end portion 69. According to another exemplary aspect, a degree of closure (including complete closure) of the passage 66 during various portions of the cardiac cycle may be selected so as to control the pressure in the coronary vein CV. As described above, these various features of the implant may be selected to control the internal pressure, for example the mean wedge pressure, of the coronary vein CV during retroperfusion and may be determined based on the measured internal pressure of the coronary vein CV before or after forming the passageway 22.

The partially-obstructing implant 65 may be self-expandable. Alternatively, the partially-obstructing implant 65 may be expandable by a dilation mechanism, such as a balloon. A balloon (not shown) disposed within the passage 66 may be expanded in the lumen 25 of the coronary vein CV to expand the partially-obstructing implant 65. The partially-obstructing implant 65 may further include an attachment mechanism, for example, in the form of a plurality of prongs 40 extending from the external surface 67, or other suitable mechanisms.

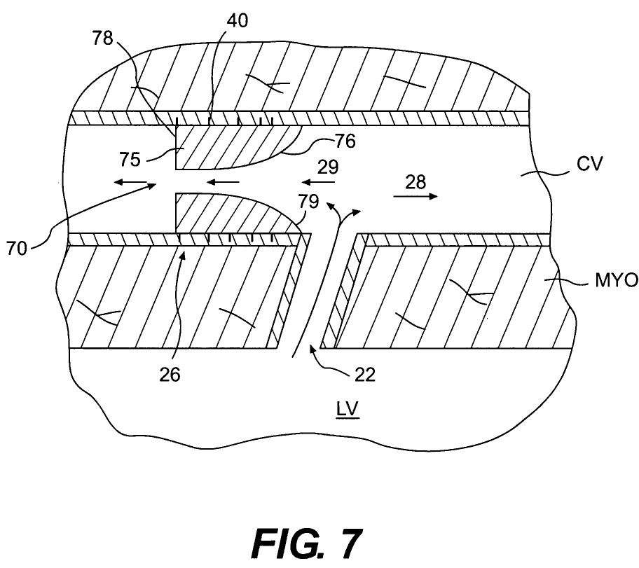

FIG. 7 illustrates another exemplary embodiment of a blocking device 70 for partially obstructing the coronary vein at a location so as to allow at least some antegrade blood flow past the location during a cardiac cycle. The embodiment illustrated in FIG. 7 is similar to the embodiment illustrated in FIG. 6, except that the implant 70 defines a passage 76 that has a non-uniform cross-section. As shown, the passage 76 is in the form of a windsock where the cross-section is smaller in the middle portion than in the end portion 79 nearest to the passageway 22. The cross-section of the passage 76 from the middle portion to the end portion 78 opposite the end portion 79 may be substantially uniform or may be tapered to gradually decrease toward the end portion 78. Of course, the tapered configuration illustrated in FIG. 7 is exemplary and other tapered configurations may be used as well. For example, the direction of taper could be opposite to that shown in FIG. 7.

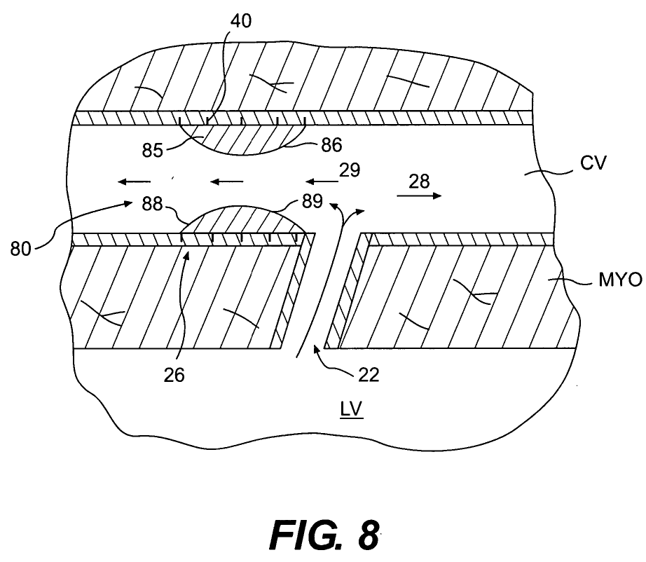

Another exemplary embodiment of a blocking device in the form of an implant defining a passage with a non-uniform cross-section is shown in FIG. 8. The blocking device 80 is configured for partially obstructing the coronary vein CV at the location 26 so as to allow at least some antegrade blood flow past the location during a cardiac cycle. In the embodiment illustrated in FIG. 8, the implant 85 defines a passage 86 that has a non-uniform cross-section where the cross-section is smaller in the middle portion than in the end portion 89 nearest to the passageway 22 and the end portion 88 opposite the end portion 89. As shown, the cross-section of the passage 86 is tapered to gradually increase from the middle portion toward the end portions 88 and 89.

These tapered configurations may provide another mechanism to control the internal pressure of the coronary vein CV during retroperfusion. Different tapered configurations may be selected so as to provide different flow resistances through the passage, for example. Accordingly, in addition to the various features explained above, a degree and/or configuration of the taper and/or passage cross-section may be used as yet another mechanism to control the internal pressure of the coronary vein CV during retroperfusion.

As with the embodiment of FIG. 6, the blocking devices 70 and 80 illustrated in FIGS. 7 and 8 may be configured to allow some antegrade blood flow therethrough throughout a cardiac cycle. For example, the passages 76 and 86 may remain open throughout the cardiac cycle. Alternatively, the passages 76 and 86 may be configured to close, e.g. by collapsing, during at least part of the cardiac cycle thereby allowing antegrade blood flow through the passage 76 and 86 during only part of the cardiac cycle. According to another exemplary aspect, a degree of closure (including complete closure) of the passages 76 and 86 during various portions of the cardiac cycle may be selected so as to control the pressure in the coronary vein CV.

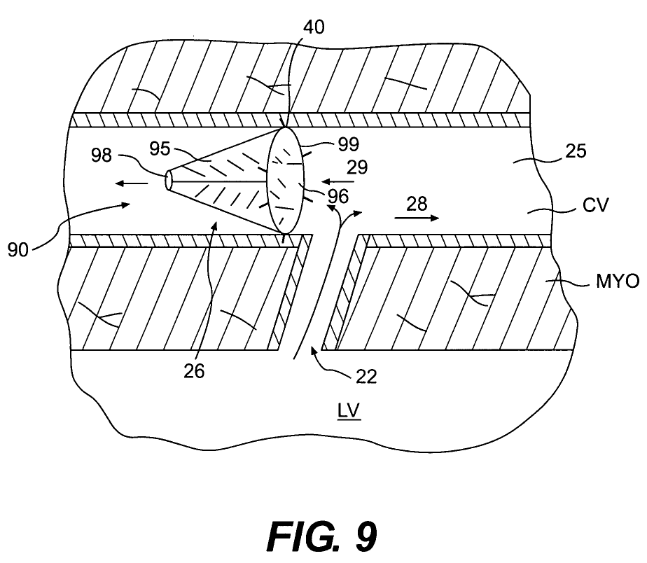

Yet another exemplary embodiment of a blocking device for partially obstructing the coronary vein at a location so as to allow at least some antegrade blood flow past the location during a cardiac cycle is shown in FIG. 9. The blocking device 90 illustrated in FIG. 9 is an implant in the form of a cone 95. As shown, the cone 95 may have a tapered passage 96 where its cross-section gradually decreases from the end portion 99 closest to the passage 22 toward the end portion 98 opposite the end portion 99. In addition to the passage 96, the body of the cone 95 itself may allow at least some antegrade blood flow past the location 26. That is, the lateral surface of the cone 95 between the ends may be covered with a porous, filter-like material that permits escape of some blood therethrough. The cone 95 also may be provided with an attachment mechanism, such as a plurality of prongs 40, for example, to secure the filter 95 within the lumen 25 of the coronary vein CV.

As with the embodiments discussed above, various configurations of the cone 95, such as the porosity of the covering of the lateral surface, the size of the openings at each end 98, 99, the degree of taper of the passage 22, etc. may be altered so as to obtain a desired antegrade flow therethrough and thereby control the pressure within the coronary vein CV.

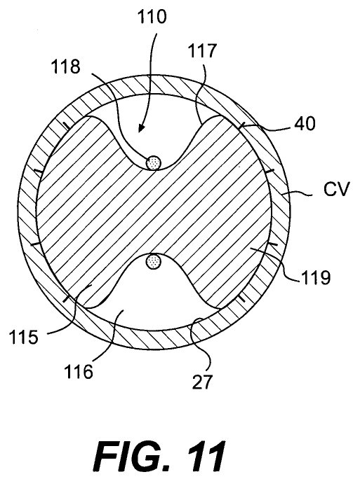

FIG. 11 illustrates yet another exemplary embodiment of a blocking device for partially obstructing the coronary vein at a location so as to allow at least some antegrade blood flow past the location during a cardiac cycle. FIG. 11 shows a cross-sectional view of the coronary vein CV at the location 26. As shown, the blocking device 110 includes an implant 115 configured to be placed within the lumen 25 of the coronary vein CV. The implant 115 may have a solid face 119 or may be substantially in the form of a solid plug, for example. In this way, blood flow through the interior of the blocking device is prevented. The blocking device 110 further includes a biasing member 118, such as a thin elastic or inelastic band, for example, along a length of the external surface 117 of the implant 115. Accordingly, while a portion of the external surface 117 of the implant 115 engages the inner surface 27 of the coronary vein CV, a space 116 is formed between another portion of the external surface 117 and the inner surface 27 of the coronary vein CV. This space 116 allows at least some antegrade blood flow past the location 26 during a cardiac cycle.

The end face 119 closest to the passageway 22 may be rigid so that it does not deform throughout the cardiac cycle. Alternatively, as described with respect to FIGS. 4A and 4B, the end face 119 may be flexible so as to deform toward the other end portion during at least a portion of the cardiac cycle. The partially-obstructing implant 115 may be self-expandable. Also, the partially-obstructing implant 115 may include an attachment mechanism, such as a plurality of prongs 40, or other attachment mechanisms.

The size of the space 116 may be varied by selecting different biasing strengths for the biasing member 118. Accordingly, in addition to the various features explained above, the biasing strength of the biasing member 118 may be selected to control the internal pressure of the coronary vein CV during retroperfusion.

As described above with respect to the embodiments of the blocking devices of FIGS. 5C-8, for example, an alternative configuration of the blocking device of FIG. 11 may be such that the implant 115 expands against the biasing member 118 to fill the entire lumen of the coronary vein CV so as to prevent antegrade blood flow past the location 26 during a portion of the cardiac cycle, such as during systole, for example. During another portion of the cardiac cycle, such as diastole for example, the pressures in the coronary vein may be such that the biasing member 118 is allowed to push in on portions of the outer surface of the implant 115 so as to allow antegrade blood flow through the space 116, thereby controlling the pressure in the coronary vein CV and permitting an efficient perfusion of the heart with the retrograde blood flow.

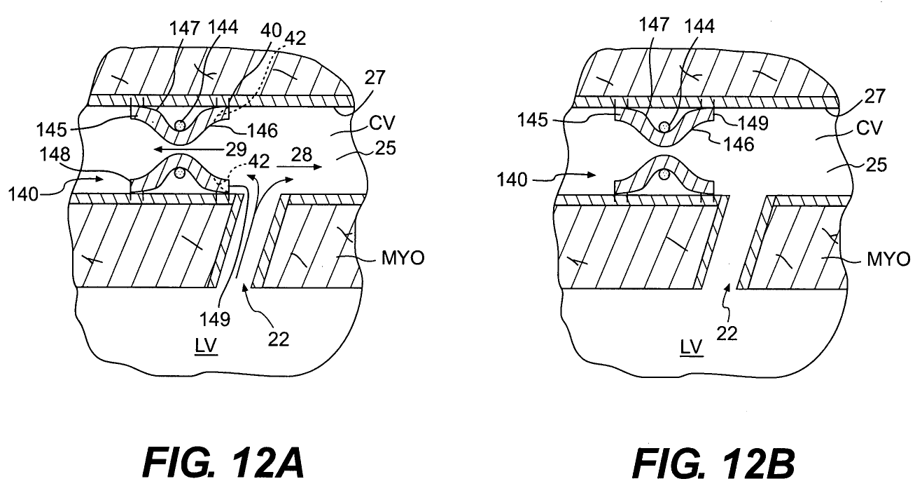

FIGS. 12A and 12B illustrate yet another exemplary embodiment of a blocking device for partially obstructing the coronary vein so as to allow at least some antegrade blood flow past the location during the cardiac cycle. For example, the blocking device 140 may allow antegrade blood flow past the location 26 during systole and prevent antegrade blood flow past the location 26 during diastole. As shown, the device 140 includes a partially-obstructing implant 145 to be placed within the lumen 25 of the coronary vein CV. The partially-obstructing implant 145 generally in the form of a conduit, which may be a stent, for example, includes an external surface 147. At least a portion of the external surface 147 is configured to engage the inner surface 27 of the coronary vein CV. The implant 145 may have openings at both ends 148 and 149.

The partially-obstructing implant 145 includes a lumen 146 extending between the two ends 148, 149 and a biasing member 144, such as, for example, a relatively thin elastic or inelastic band, may be disposed around the external surface 147. For example, the biasing member 148 may be disposed around a middle portion of the implant 145. The biasing strength and elasticity of the biasing member 144 may be selected such that it causes the lumen 146 to open during a first portion of the cardiac cycle, as shown in FIG. 12A, and to close during a second portion of the cardiac cycle, as shown in FIG. 12B. Accordingly, in addition to the various other features explained above, the biasing strength of the biasing member 144 may be selected so as to control the amount of antegrade blood flow through lumen 146 and thus the internal pressure of the coronary vein CV during retroperfusion.

The partially-obstructing implant 145 may be self-expandable. Alternatively, the partially-obstructing implant 145 may be balloon-expandable and/or expandable by other dilation mechanisms. The biasing member 144 may be elastic or inelastic. Also, in the case of a balloon-expandable implant, the degree of closure may be controlled by making the biasing member 144 more compliant under different balloon expansion pressures. In other words, the biasing member 144 may be configured such that its elasticity or compliance have different values depending on the different pressure applied to expand the implant 145. This may permit the biasing member 144 to be elastic so as to expand to a certain degree and then to be substantially inelastic, i.e., resist further expansion, once expanded to the desired degree. The partially-obstructing implant 145 may further include an attachment mechanism, such as, for example, a plurality of prongs 40 extending from the external surface 147.

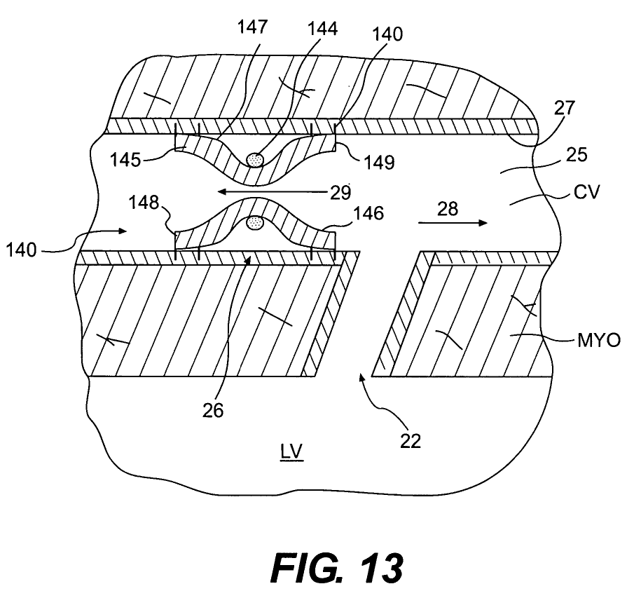

As illustrated in FIG. 13, the embodiment shown in FIGS. 12A and 12B may be modified such that it allows at least some antegrade blood flow past the location 26 throughout the cardiac cycle. In other words, by selecting the appropriate biasing strength of the biasing member 144, the passage 146 may remain open during both HENDERSON systole and diastole. As an example, the biasing member 144 may be in the form of a substantially non-compliant band that does not substantially change after the implant's expansion, as explained above.

As described above, the biasing member 144 may be elastic and made from materials such as elastomers, rubbers, silicones, expandable metals, and other materials exhibiting elastic behavior. Alternatively, the biasing member 144 may be substantially inelastic and made from materials such as, for example, metals, such as stainless steel, for example, rigid plastics, such as polyethylene, for example, and other substantially inelastic materials. In an exemplary embodiment, the biasing member 144 may be in the form of a suture encircling the implant.

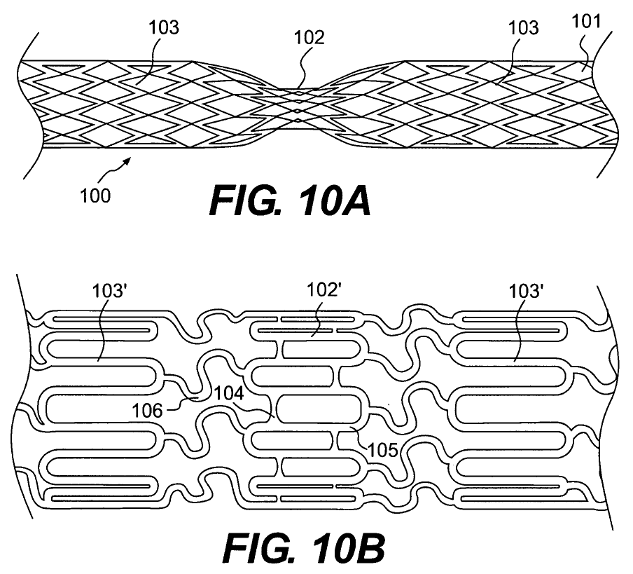

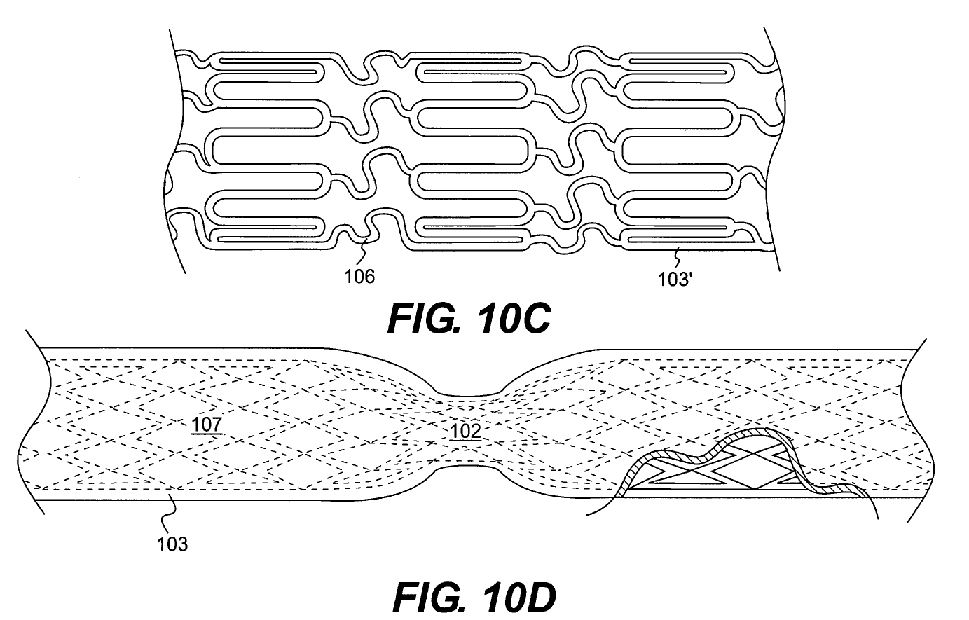

FIGS. 10A-10D show an exemplary embodiment of an implant 100 having a restricted region 102 similar to the embodiment of FIGS. 12A, 12B, and 13, except that the implants in FIGS. 10A-10D have no biasing member. Rather, implant 100 may be in the form of a stent 101, for example, and may have a structure such that, upon expansion of the stent 101 within the vein, a portion of the stent 101 expands less than other portions 103 of the stent 101 such that the size of the lumen is restricted at the region 102. To form the cell structure of the stent 101 having a restricted region 102, methods such as electrochemical etching and/or laser cutting of a single piece tube, for example, and other methods for making expandable stents may be used. However, the cell structure that forms the restricted region 102 of the stent 101 differs from the cell structures that form the nonrestricted regions 103 of the stent 101. The different cell structure that is used to form the restricted region 102 is such that, upon expansion of the stent 101, the restricted region 102 expands less than the other portions 103 of the stent.

FIGS. 10B and 10C show stent 101 prior to expansion. Prior to expansion, the stent 101 may have a substantially constant diameter over its length. Upon expansion of the stent 101, the restricted stent cell structure 102′ expands less than the remainder of the stent formed of the expandable stent cells structure 103′ so as to provide the stent 101 with a restricted region 102 in which the size of the lumen decreases. In an exemplary embodiment, the diameter of the restricted region 102 after expansion of the stent 101 may be approximately the same as the diameter of the stent 101 prior to expansion. In another embodiment, after expansion of the stent 101, stent cell structure 102′ may be configured so that the restricted region 102 has a diameter slightly larger than the diameter of the stent 101 prior to expansion.

According to an exemplary embodiment, the stent 101 may have at least two differing stent cell structures. For example, a first stent cell structure 102′ may make up the restricted region 102 of the stent 101, as shown in FIG. 10B, for example, and a second stent cell structure 103′ may form the expanded (e.g., nonrestricted) portions 103 of the stent 101, as shown in FIG. 10C, for example. The restricted region 102 may be formed of a single stent cell, as shown in FIGS. 10A and 10B, for example. However, any number of stent cells may be used to form the restricted region 102. Moreover, there may be a plurality of restricted regions along the length of the stent, and those regions may be spaced from one another as desired. In the exemplary embodiment of FIG. 10B, the stent cell structure 102′ includes a repeating wave-like pattern that traverses the circumference of the stent 101. Struts 104 connect adjacent legs 105 forming the wave-like pattern. The struts 104 are configured so as to prevent, or at least hinder, the expansion of the stent cell structure 102′ in the radial direction. FIGS. 10B and 10C show an exemplary embodiment of the stent cell structure 103′ which forms the nonrestricted regions 103 of the stent 101. The stent cell structure 103′ also includes stent cells formed of a repeating wave-like pattern. However, stent cell structure 103′ lacks struts between the legs of the wave-like pattern, so as to permit the stent cell structure 103′ to expand in a radial direction. In the exemplary embodiment of FIGS. 1A-10C, the various stent cells forming the stent 101 are connected to one another via articulations 106.

The specific stent cell structures shown in FIGS. 10A-10C are exemplary only, and other cell structures that allow the implant to expand more in certain regions than in other regions may be utilized and are considered within the scope of the invention. For example, the number, placement, size, shape, material, and/or other characteristics of the struts may be altered as desired in order to provide greater or lesser expansion of the restricted stent cell structure 102′.

Instead of forming the differing stent cell structures from a single piece tube, as described above with reference to FIGS. 1A-1C, the restricted region stent cell structure may be formed from a tube that is separate from the tubes which form the non-restricted regions. In this approach, the restricted region stent cell structure may be joined, for example by welding, adhesive, or other joining mechanisms with the stent cell structures forming the non-restricted regions 103, in an end-to-end configuration so as to form a substantially continuous stent structure. Alternatively, a wire stent could be used and a portion of the wire stent could be formed into the restricted region. This could be accomplished, for example, by welding or otherwise attaching together overlying or neighboring wires to each other in such a way so as to prevent expansion of the attached wires.

Stent 101 may be made of materials such as, for example, stainless steel, shape memory alloys, metals, plastics, and other materials used for making stents. In an exemplary embodiment, the stent may be provided with a covering 107, as shown in FIG. 10D. The covering 107 may cover an outer surface and/or an inner surface of the stent 101. Such a covering 107 may be a made of expanded polytetrafluoroethylene (ePTFE), polyethylene terephthalate (PET), Dacron, and other coverings used for stents and other medical devices, for example. Moreover, in an exemplary aspect, a coating, such as heparin, for example, may be used in conjunction with the covering, and may be placed over the covering on an inner and/or an outer surface of the stent 101.

As with other exemplary embodiments described herein, the implant of FIGS. 10A-10D may be self-expandable or expandable by a dilation mechanism, such as a balloon, for example. Also, the implant may be provided with attachment mechanisms, such as barbs, hooks, tabs, prongs, sutures, and the like, to assist in securing the implant in position within the coronary vein.

Further, it is contemplated that the restricted region 102 of the stent 101 may be such that the lumen of the stent 101 is completely or substantially collapsed during either a portion of or throughout the cardiac cycle. For example, in the case of the latter, the stent 101 may be a self-expandable stent, with the restricted region 102 being nonexpandable such that the lumen remains completely or substantially collapsed at that region upon deployment of the stent 101. In the case of the former, the stent structure 102′ at the restricted region 102 may be configured so as to provide some expansion to open the stent lumen during a portion of the cardiac cycle while closing or substantially closing the stent lumen during another portion of the cardiac cycle. As an example, the expansion of the restricted region may occur by providing a stent structure that responds to various forces associated with the cardiac cycle.

FIGS. 14A and 14B illustrate yet another exemplary embodiment of a device for partially obstructing the coronary vein so as to allow at least some antegrade blood flow past the location during a cardiac cycle. For example, the blocking device 160 may allow antegrade blood flow past the location 26 during diastole and prevent antegrade blood flow past the location 26 during systole. As shown, the device 160 includes a partially-obstructing implant 165 to be placed within the lumen 25 of the coronary vein CV. Similar to the configuration of FIGS. 12A and 12B, the partially-obstructing implant 165 includes an external surface 167 configured to engage the inner surface 27 of the coronary vein CV and two open ends 168, 169.

The partially-obstructing implant 165 defines a passage 166 extending between the open ends 168, 169. One or more inflatable members 164 may be disposed adjacent the external surface 167 of the implant. The inflatable members 164 may have a variety of forms, such as, for example, balloons. An exemplary form of the inflatable members 164 may be a pocket 163 containing an air bubble 162, as shown in FIGS. 14A and 14B. The pocket 163 may be formed between the external surface 167 and the passage 166. Alternatively, the pocket 163 may form a portion of the external surface of the implant 165. The air bubble 162 may be suspended in an appropriate substance which fills the pocket 163. The size and/or the number of the air bubbles 162 in each pocket 163 may be selected such that the air bubbles 162 at least partially collapse to open the passage 166 during a first portion of the cardiac cycle, as shown in FIG. 14A, and regain their original shape to close the passage 166 during a second portion of the cardiac cycle, as shown in FIG. 14B. Also, the number of pockets 163 containing air bubbles 162 as well as the distribution of such pockets around the implant 165 may also be chosen so as to control the opening and closing of the passage 166. Any combination of the number, size, and distribution of the air bubbles 162 and/or the pockets 163 may be selected so as to control the opening and closing of the passage 166 or to control the degree of the opening of the passage 166. By way of example, to control the opening and closing of the passage 166 based on the cardiac cycle phases, an external inflation source (not shown) may be provided and may be either manually or automatically actuated so as to inflate and deflate the air bubbles 162.

Accordingly, in addition to the various features explained above, any combination of the number, size, and distribution of the air bubbles 162 and/or the pockets 163 may be selected to control amount of occlusion of the coronary vein CV, thereby controlling the amount of antegrade flow through the passage 166 and the internal pressure of the coronary vein CV during retroperfusion. As described above, these features to control the internal pressure of the coronary vein CV during retroperfusion may be selected based on the measured internal pressure of the coronary vein CV before or after forming the passageway 22.

The partially-obstructing implant 165 may be self-expandable. Alternatively, the partially-obstructing implant 165 may be expandable by a dilation mechanism. For example, a balloon disposed within the passage 166 may be expanded in the lumen 25 of the coronary vein CV to expand the partially-obstructing implant 165. The partially-obstructing implant 165 may also be provided with an attachment mechanism, such as the plurality of prongs 40 shown in FIGS. 14A and 14B, for example, to secure the implant 165 within the coronary vein CV.

As illustrated in FIG. 15, the embodiment shown in FIGS. 14A and 14B may be modified such that it allows at least some antegrade blood flow past the location throughout the cardiac cycle. In other words, by selecting appropriate combination of the number, size, and distribution of the air bubbles 162 and/or the pockets 163, the passage 166 may remain open during both systole and diastole. In the embodiment wherein the passage 166 remains open throughout the cardiac cycle, each cross-section of the passage 166 may remain the same throughout the cardiac cycle or may be varied during the cardiac cycle based on pressures associated with the various portions of the cardiac cycle.

As discussed, the various exemplary embodiments of blocking devices described above may be used to treat the heart via retroperfusion. In an exemplary aspect, the wedge pressure, for example the mean wedge pressure, of the coronary vein of a patient may be measured, for example prior to forming a passage between an oxygenated blood-containing anatomical structure and the coronary vein to establish retrograde flow in the coronary vein. Based on the measured wedge pressure, a target pressure or range of pressures may be determined. For example, the target pressure or range of pressures may be selected so that the pressure in the coronary vessel is high enough to cause retroperfusion but low enough so that the heart is not overperfused and/or so that the risk of damage to the vessel due to excess pressure is minimized. Based on the measured pressure and/or the target pressure or range of pressures, a blocking device may be selected so as to control the pressure in the coronary vein to approximately the target level. Factors such as the amount of obstruction, location of the obstruction, and/or amount of pressure absorption also may be determined based on the measured pressure so as to control pressure in the coronary vessel to the predetermined target level and those factors may be used to determine which of a plurality of blocking devices having differing characteristics to select for implantation. In this way, for each patient, the type and/or location of the blocking device used may be tailored to meet that patient's particular needs, thereby optimizing treatment of the patient.

An exemplary embodiment of a system for use in retroperfusing a patient's heart may therefore include a pressure measuring device for measuring the wedge pressure of the coronary vein and at least one implant configured to be positioned relative to the coronary vein and to at least partially block antegrade flow in the coronary vein. As an example, such a system may include a plurality of implants, for example in the form of the various blocking devices described herein, and each of the implants may have differing characteristics so that they partially obstruct the flow in differing ways.

It should be understood that the various embodiments described herein are exemplary only. Various other structural configurations may be envisioned and provide the same function for at least partially obstructing antegrade blood flow through the coronary vein. Further, embodiments of blocking devices that have been described as permitting at least some antegrade blood flow past the location also may be configured so as to completely block the coronary vein CV so as to prevent antegrade blood flow past the location of the blocking device throughout the cardiac cycle.

Additionally, in its broadest aspects, the invention is not limited to retroperfusing a heart in the manner described with reference to the exemplary embodiments. The devices and methods described may be used to control fluid flows and pressures in a variety of other settings, as will be apparent to those skilled in the art. For example, the devices and methods may be useful to retroperfuse organs and tissue regions other than those associated with the heart. Further, the devices and methods have been discussed in conjunction with retroperfusing the venous system via oxygenated blood-containing anatomical structures. However, it is envisioned that the devices and methods also may be used in conjunction with retroperfusing the venous system via sources other than oxygenated blood-containing anatomical structures. Such sources may include, for example, artificial sources or external sources which can be placed in flow communication with the venous system to provide retrograde flow therethrough. In addition, the devices and methods described herein may find use in the field of flow control during the perfusion of various organs and other portions of the body, in which case the various partial blocking devices and methods may be used in conjunction with the arterial system to control blood flow and/or pressures therein.

Other embodiments of the invention will be apparent to those skilled in the art from consideration of the specification and practice of the invention disclosed herein. The specification and examples are exemplary only and the present invention is intended to cover modifications and variations.

1. A method of treating a heart, comprising:
measuring an internal pressure of a coronary vein so as to determine a measured internal pressure;
flowing blood through a passageway between a blood-containing anatomical structure and the coronary vein so as to allow retrograde blood flow in the coronary vein; and
based on the measured internal pressure, at least partially obstructing the coronary vein at a location upstream of the passageway relative to a direction of the retrograde blood flow.

2. The method of claim 1, further comprising selecting an amount of obstruction based on the measured internal pressure.

3. The method of claim 1, wherein the measuring of the internal pressure of the coronary vein is performed before the flowing of the blood through the passageway.

4. The method of claim 1, further comprising selecting the location upstream of the passageway based on the measured internal pressure.

5. The method of claim 1, wherein the at least partially obstructing the coronary vein includes allowing at least some antegrade blood flow past the location during at least a portion of a cardiac cycle.

6. The method of claim 1, wherein the at least partially obstructing the coronary vein includes allowing at least some antegrade blood flow past the location throughout a cardiac cycle.

7. The method of claim 1, wherein the at least partially obstructing the coronary vein includes allowing at least some antegrade blood flow past the location during diastole and preventing antegrade blood flow past the location during systole.

8. The method of claim 1, wherein the at least partially obstructing the coronary vein includes completing obstructing the coronary vein to prevent antegrade blood flow past the location throughout a cardiac cycle.

9. The method of claim 1, further comprising placing an implant at the location, wherein the implant is configured to at least partially obstruct the coronary vein.

10. The method of claim 9, further comprising selecting the implant from a plurality of implants based on the measured internal pressure.

11. The method of claim 9, wherein the implant is configured to completely obstruct the coronary vein during at least a portion of a cardiac cycle.

12. The method of claim 11, wherein the implant is configured to allow at least some antegrade blood flow toward the location during at least a portion of a cardiac cycle.

13. The method of claim 11, wherein the implant is configured to at least partially absorb pressure in the coronary vein.

14. The method of claim 9, wherein the implant is configured to allow at least some antegrade blood flow past the location during a portion of a cardiac cycle.

15. The method of claim 14, wherein the implant is configured to allow at least some antegrade blood flow past the location during diastole and prevent antegrade blood flow past the location during systole.

16. The method of claim 9, wherein the implant is configured to completely obstruct the coronary vein throughout a cardiac cycle.

17. The method of claim 9, wherein the implant defines a lumen configured to allow at least some antegrade blood flow therethrough.

18. The method of claim 9, wherein the implant is configured to allow at least some antegrade blood flow through a space formed between an external surface of the implant and an inner surface of the coronary vein.

19. The method of claim 9, wherein the placing of the implant includes placing the implant within the lumen of the coronary vessel.

20. The method of claim 19, further comprising expanding the implant.

21. The method of claim 20, wherein the expanding of the implant includes expanding the implant via a balloon.

22. The method of claim 9, wherein at least part of the implant is configured to deform.

23. The method of claim 22, wherein the implant comprises an end face configured to deform.

24. The method of claim 1, further comprising at least partially collapsing the coronary vein at the location.

25. The method of claim 24, wherein the at least partially collapsing includes completely collapsing the coronary vein during at least a portion of a cardiac cycle.

26. The method of claim 24, wherein the at least partially collapsing includes completely collapsing the coronary vein during systole.

27. The method of claim 24, wherein the at least partially collapsing includes completely collapsing the coronary vein throughout a cardiac cycle.

28. The method of claim 24, wherein the at least partially collapsing includes partially collapsing the coronary vein throughout a cardiac cycle.

29. The method of claim 1, further comprising placing magnetic elements at the location so as to partially collapse the coronary vein at the location.

30. The method of claim 1, wherein the blood-containing anatomical structure is a heart chamber.

31. The method of claim 30, wherein the heart chamber is a left ventricle.

32. The method of claim 1, wherein the blood-containing anatomical structure is a coronary artery.

33. The method of claim 1, further comprising placing a conduit in the passageway.

34. The method of claim 1, further comprising forming the passageway.

35. The method of claim 1, wherein measuring the internal pressure includes measuring the mean wedge pressure.

36. A method of retroperfusion, comprising:
measuring an internal pressure of a portion of the venous system to determine a measured internal pressure;
flowing blood through a passageway between a blood-containing anatomical structure and the portion of the venous system so as to allow retrograde blood flow in the portion of the venous system; and
based on the measured internal pressure, at least partially obstructing the portion of the venous system at a location upstream of the passageway relative to a direction of the retrograde blood flow.

37. The method of claim 36, wherein measuring the internal pressure includes measuring the mean wedge pressure.

38. A method of treating a heart, comprising:
flowing blood through a passageway between a heart chamber and a coronary vein so as to cause retrograde blood flow in the coronary vein; and
at least partially obstructing the coronary vein at a location upstream of the passageway relative to a direction of the retrograde blood flow.

39. The method of claim 38, wherein the at least partially obstructing the coronary includes allowing at least some antegrade blood flow past the location during at least a portion of a cardiac cycle.

40. The method of claim 38, wherein the at least partially obstructing the coronary vein includes allowing at least some antegrade blood flow past the location throughout the cardiac cycle.

41. The method of claim 38, wherein the at least partially obstructing the coronary vein includes allowing at least some antegrade blood flow past the location during diastole and preventing antegrade blood flow past the location during systole.

42. The method of claim 38, wherein the at least partially obstructing the coronary vein includes completely obstructing the coronary vein so as to prevent antegrade blood flow past the location throughout a cardiac cycle.

43. The method of claim 38, further comprising measuring a mean wedge pressure of the coronary vein to determine a measured mean wedge pressure and at least partially obstructing the coronary vein based on the measured mean wedge pressure.

44. The method of claim 38, further comprising measuring a mean wedge pressure of the coronary vein to determine a measured mean wedge pressure and selecting the location upstream of the passageway based on the measured mean wedge pressure.

45. A system for use in treating a heart by causing retrograde blood flow in a coronary vein, the system comprising:
a pressure measuring device configured to measure an internal pressure of a coronary vein; and
at least one implant configured to be placed relative to the coronary vein at a location upstream, relative to a direction of the retrograde blood flow, of a passageway formed between the coronary vein and a blood-containing anatomical structure,
wherein the implant is configured to at least partially obstruct the coronary vein.

46. The system of claim 45, wherein the at least one implant is configured to allow at least some antegrade blood flow in the coronary vein past the location during at least a portion of a cardiac cycle.

47. The system of claim 46, wherein the at least one implant is configured to allow at least some antegrade blood flow past the location throughout the cardiac cycle.

48. The system of claim 46, wherein the at least one implant is configured to allow at least some antegrade blood flow past the location during diastole and prevent antegrade blood flow past the location during systole.

49. The system of claim 45, wherein the at least one implant is configured to prevent antegrade blood flow past the location throughout a cardiac cycle.

50. The system of claim 45, wherein the at least one implant is configured to prevent antegrade blood flow past the location during at least part of a cardiac cycle.

51. The system of claim 45, wherein the at least one implant is configured to be placed within a lumen of the coronary vein at the location.

52. The system of claim 45, wherein the at least one implant defines a passage configured to allow at least some antegrade blood flow therethrough during at least a portion of a cardiac cycle.

53. The system of claim 52, wherein the passage has a substantially uniform cross-section.

54. The system of claim 52, wherein the passage has a non-uniform cross-section.

55. The system of claim 54, wherein the cross-section is smaller in a middle portion of the passage than in an end portion of the passage.

56. The system of claim 55, wherein the end portion is an end that faces the passageway.

57. The system of claim 54, wherein the passage is at least partially tapered.

58. The system of claim 52, wherein the passage is configured to close during at least a portion of the cardiac cycle.

59. The system of claim 58, wherein the passage is configured to close throughout the cardiac cycle.

60. The system of claim 52, wherein the passage is configured to at least one of expand and contract during the cardiac cycle.

61. The system of claim 52, further comprising a biasing member around an external surface of the at least one implant.

62. The system of claim 61, wherein the biasing member is configured to reduce a cross-section of the passage.

63. The system of claim 61, wherein the biasing member is configured to at least partially collapse a portion of the at least one implant.

64. The system of claim 51, wherein the at least one implant includes a filter.

65. The system of claim 51, wherein the at least one implant includes an external surface, at least a portion of the external surface being configured to engage an inner surface of the coronary vein.

66. The system of claim 65, wherein a space is formed between at least an additional portion of the external surface of the at least one implant and the inner surface of the coronary vein, the space being configured to allow at least some of the antegrade blood flow therethrough.

67. The system of claim 51, wherein the at least one implant is expandable.

68. The system of claim 67, wherein the at least one implant is self-expandable.

69. The system of claim 67, wherein the at least one implant is expandable via a dilation mechanism.

70. The system of claim 69, wherein the dilation mechanism is a balloon.

71. The system of claim 45, wherein the at least one implant includes a flexible portion configured to deform during at least a portion of a cardiac cycle.

72. The system of claim 71, wherein the flexible portion is an end face of the at least one implant.

73. The system of claim 72, wherein the end face faces toward the passageway.

74. The system of claim 73, wherein the end face is configured to prevent antegrade blood flow past the location.

75. The system of claim 71, wherein the flexible portion is configured to deform to absorb pressure in the coronary vessel.

76. The system of claim 45, further comprising an attachment mechanism associated with the at least one implant.

77. The system of claim 76, wherein the attachment mechanism is configured to secure the at least one implant within a lumen of the coronary vein.

78. The system of claim 52, further comprising at least one mechanism configured to act on an external portion of the at least one implant so as to at least partially collapse the at least one implant proximate the at least one mechanism.

79. The system of claim 78, wherein the at least one mechanism includes an inflatable member.

80. The system of claim 79, wherein the at least one mechanism includes an air bubble.

81. The system of claim 78, wherein the at least one mechanism includes a biasing member.

82. The system of claim 78, wherein the at least one mechanism is configured to collapse the at least one implant such that the passage closes during a portion of the cardiac cycle.

83. The system of claim 45, wherein the at least one implant includes magnetic elements configured to at least partially collapse the coronary vessel at the location during at least a portion of the cardiac cycle.

84. The system of claim 83, wherein the magnetic elements are configured to be implanted in a heart wall surrounding the coronary vessel.

85. The system of claim 83, wherein the magnetic elements are configured to completely collapse the coronary vessel at the location during at least part of a cardiac cycle.

86. The system of claim 85, wherein the magnetic elements are configured to completely collapse the coronary vessel at the location during systole.

87. The system of claim 83, wherein the magnetic elements are configured to at least partially collapse the coronary vessel throughout the cardiac cycle.

88. The system of claim 45, wherein the passageway is configured to flow blood between the blood-containing anatomical structure and the coronary vein so as to cause the retrograde blood flow in the coronary vein.

89. The system of claim 45, wherein the blood-containing anatomical structure is a heart chamber.

90. The system of claim 45, wherein the heart chamber is a left ventricle.

91. The system of claim 45, wherein the blood-containing anatomical structure is a coronary artery.

92. The system of claim 45, wherein the at least one implant includes a plurality of implants having differing characteristics.

93. The system of claim 54, wherein the implant comprises a stent having differing stent structures such that a first portion of the stent defines a lumen that is smaller than a second portion of the stent.

94. The system of claim 93, wherein the stent is expandable and the first portion of the stent is configured to expand less than the second portion of the stent.

95. A device for use in treating a heart by causing retrograde blood flow in a coronary vein, the device comprising:
an implant configured to be placed within the coronary vein at a location upstream, relative to a direction of the retrograde blood flow, of a passageway between the coronary vein and a heart chamber,
wherein the implant is configured to at least partially obstruct the coronary vein.

96. The device of claim 95, wherein the implant is configured to control an internal pressure of the coronary vein.

97. The device of claim 95, wherein the implant is configured to partially obstruct the coronary vein at the location.

98. The device of claim 95, wherein the implant is configured to completely obstruct the coronary vein at the location.

99. The device of claim 95, wherein the implant is configured to permit at least some antegrade blood flow in the coronary vein past the location.

100. The device of claim 99, wherein the implant is configured to prevent antegrade blood flow past the location during systole and allow antegrade blood flow past the location during diastole.

101. The device of claim 99, wherein the implant is configured to permit at least some antegrade blood flow past the location throughout a cardiac cycle.

102. The device of claim 95, wherein the implant is configured to prevent antegrade blood flow past the location throughout a cardiac cycle.

103. The device of claim 96, wherein the implant is configured to relieve excess internal pressure in the coronary vein.

104. The device of claim 96, wherein the implant is configured to maintain the internal pressure approximately within a predetermined range.

105. The device of claim 45, wherein the implant is configured to relieve excess pressure in the coronary vein.

106. The device of claim 45, wherein the implant is configured to maintain the internal pressure approximately within a predetermined pressure range.

107. The method of claim 1, wherein the at least partially obstructing the coronary vein includes maintaining the internal pressure approximately within a predetermined pressure range.

108. The method of claim 38, wherein at least partially obstructing the coronary vein includes at least partially obstructing the coronary vein so as to control an internal pressure of the coronary vein.

109. A system for use in retroperfusion, the system comprising:
a pressure measuring device configured to measure an internal pressure of a portion of the venous system; and
at least one implant configured to be placed relative to the portion of the venous system at a location upstream, relative to a direction of the retrograde blood flow, of a passageway formed between the portion of the venous system and a blood-containing anatomical structure,
wherein the implant is configured to at least partially obstruct the portion of the venous system.

110. The system of claim 109, wherein the internal pressure is mean wedge pressure.

111. A stent comprising:
at least a first cell structure and at least a second cell structure that differs from the first cell structure,
wherein the stent is expandable and the first cell structure is configured so as to expand less than the second cell structure upon expansion of the stent.

112. The stent of claim 111, wherein the first cell structure comprises a series of repeating wave-like segments traversing a circumference of the stent and a plurality of struts connecting the segments so as to at least substantially hinder radial expansion of the first cell structure.
